(19) United States
(12) Patent Application Publication
(10) Pub. No.: US 2025/0339647 A1
(43) Pub. Date: Nov. 6, 2025

(54) INFUSION SET AND AN ADHESIVE STRUCTURE THEREFOR

(71) Applicant: Unomedical A/S, Lejre (DK)

(72) Inventors: Lisa Jaunet Van Kooten, Copenhagen (DK); Kristoffer Hendel, Lejre (DK)

(21) Appl. No.: 19/267,009

(22) Filed: Jul. 11, 2025

Related U.S. Application Data
(63) Continuation of application No. PCT/EP2024/050680, filed on Jan. 12, 2024.
(60) Provisional application No. 63/438,861, filed on Jan. 13, 2023.

(30) Foreign Application Priority Data
Feb. 21, 2023 (GB) 2302446.6

(51) Int. Cl. A61M 25/02; A61L 24/00; A61M 5/142

(52) U.S. Cl. CPC A61M 25/02; A61L 24/0031; A61M 5/14248; A61M 2025/0266

(57) ABSTRACT

An adhesive structure for an infusion set comprises a layer of adhesive having at least one opening therethrough for receiving a cannula of an infusion set and a release liner. The layer of adhesive is a hydrocolloid adhesive, which is a blend of one or more pressure sensitive adhesives and one or more water dispersible hydrocolloids. An adaptor is provided which includes the adhesive structure; wherein the layer of adhesive has a skin-facing surface and an opposite infusion hub-facing surface; wherein a release liner is provided on the skin-facing surface; and wherein a protective layer is provided on the infusion set-facing surface. An infusion hub of an infusion set is also described, which includes the adhesive structure.

The invention relates to an infusion hub and an infusion set for subcutaneous infusion of a therapeutic agent into a patient; as well as to an adhesive structure for an infusion set and an adhesive adaptor for an infusion set. The invention also relates to patch pumps and continuous glucose monitors. Particularly, though not exclusively, the invention relates to infusion sets for subcutaneous infusion of insulin, heparin, apomorphine, arbidopa, or levodopa and/or levodopa products into a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three to four times a day (depending on, inter alia, the type of diabetes and blood sugar levels of the patient). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and requires fewer needle sticks.

The second mode is known as continuous subcutaneous insulin infusion (CSII) therapy. Infusion pump systems for CSII therapy may be worn by the patient. The systems typically include a combined infusion pump and reservoir for containing an insulin drug, for example human insulin or analogue insulin, and an insulin infusion set. The infusion set may include a cannula (for example, a polymeric catheter or metal needle) for insertion subcutaneously into the patient and flexible tubing for fluidly connecting the cannula to the reservoir. Once the cannula is inserted into the patient, it may remain in place for a period of time, i.e., days, to allow for continuous delivery of the insulin drug. The current recommended wear time for insulin infusion sets is two to three days, to avoid problems that may arise relating to the infusion set itself or to the infusion site. However, such problems may still arise within recommended wear times, resulting in early removal of the infusion set and more frequent site rotation across infusion sites (for example buttocks, abdomen and arms).

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their infusion pumps, resulting in poorer patient outcomes.

Infusion hubs of infusion sets typically include a housing, which is connected to the tubing connector in use. Infusion hubs typically include a skin-facing side provided with an adhesive to adhere the infusion hub to the patient's skin. The adhesives used to adhere the infusion hub to the skin are almost exclusively acrylic adhesives. It is considered that these adhesives may be unsuitable for longer wear times, at least for some patients, for example existing adhesives may cause irritation when left in place for longer periods of time; may become unstuck when left in place for longer periods; or may become difficult to unstick when left in place for longer periods.

Similar considerations apply to other similar devices for subcutaneous infusion, for example patch pumps, and to continuous glucose monitors (CGM) which have one or more sensors which are inserted subcutaneously in much the same way as the cannula of an infusion hub.

It is an object of embodiments of the invention to provide an improved adhesive structure for an infusion set that attempts to increase wear times of infusion sets, and/or at least mitigate one or more problems associated with known arrangements.

### BRIEF SUMMARY OF THE DISCLOSURE

The invention is defined by the appended claims. Any of the optional features outlined below apply equally to the claims, unless incompatible.

In broad terms, the present disclosure provides an adhesive structure for an infusion set, a patch pump or a continuous glucose monitor including a hydrocolloid adhesive.

The present disclosure provides an adhesive structure for an infusion set (or for a patch pump or a continuous glucose monitor). The adhesive structure may comprise a layer of adhesive. The layer of adhesive may have at least one opening therethrough for receiving a cannula of an infusion set or patch pump, or a sensor of a continuous glucose monitor. The adhesive structure may comprise a release liner. The layer of adhesive may be a hydrocolloid adhesive.

In a first aspect of the present disclosure, there is provided an adhesive structure for an infusion set; the adhesive structure comprising a layer of adhesive having at least one opening therethrough for receiving a cannula of an infusion set and a release liner; wherein the layer of adhesive is a hydrocolloid adhesive.

Hydrocolloid adhesives, have been used in other fields of technology for many years. For example, U.S. Pat. No. 4,793,337 filed in 1986 proposes wound dressings incorporating a hydrocolloid adhesive, which are considered particularly suitable for wounds emitting large amounts of fluid. Moreover, wound dressings incorporating hydrocolloid adhesives have found commercial success, for example in the DuoDERM (RTM) Extra Thin dressing, available from ConvaTec Limited of Deeside, UK, which is intended to be used for lightly exuding wounds, and which according to the instructions for use, should be replaced within 7 days and in practice are often replaced more frequently than that.

Surprisingly, this improved adhesive structure is suitable for adhering an infusion hub to the skin for extended periods of time, without causing irritation; becoming unstuck, or becoming too difficult to remove. For example, testing has shown that the improved adhesive structure can be used for an extended period of time of more than 7 days, such as an extended period of time 8-10 days, 10-14 days, and even an extended period of time of 14 or more days.

In some cases the hydrocolloid adhesive is a blend of one or more pressure sensitive adhesives and one or more water dispersible hydrocolloids.

In some cases the one or more water dispersible hydrocolloids are selected from the group consisting of guar gum, sodium carboxymethylcellulose, calcium carboxymethylcellulose, pectin, gelatin, locust bean gum, collagen, gum karaya, alginic acid, calcium alginate, and sodium alginate.

In some cases the one or more water dispersible hydrocolloids are selected from the group consisting of sodium carbomethylcellulose, pectin and gelatin.

In some cases the hydrocolloid adhesive includes two or more water dispersible hydrocolloids the group consisting of guar gum, sodium selected from carboxymethylcellulose, calcium carboxymethylcellulose, pectin, gelatin, locust bean gum, collagen, gum karaya, alginic acid, calcium alginate, and sodium alginate.

In some cases the hydrocolloid adhesive includes two or more water dispersible hydrocolloids selected from the group consisting of sodium carbomethylcellulose, pectin and gelatin.

In some cases the hydrocolloid adhesive includes three or more water dispersible hydrocolloids selected from the group consisting of guar gum, sodium carboxymethylcellulose, calcium carboxymethylcellulose, pectin, gelatin, locust bean gum, collagen, gum karaya, alginic acid, calcium alginate, and sodium alginate.

In some cases the hydrocolloid adhesive includes three or more water dispersible hydrocolloids including sodium carbomethylcellulose, pectin and gelatin.

The hydrocolloid adhesive may include from about 10 percent to about 65 percent by weight of water dispersible hydrocolloid materials.

Preferably, the water dispersible hydrocolloids are present at from about 30 percent to about 65 percent by weight of the hydrocolloid adhesive.

In some cases said pressure sensitive adhesives comprise one or more elastomeric materials selected from the group consisting of natural rubber, silicone rubber, and low molecular weight polyisobutylenes and optionally one or more thermoplastic elastomers selected from the group consisting of medium molecular weight polyisobutylenes, butyl rubber, and styrene compolymers.

In some cases said pressure sensitive adhesives include natural or synthetic viscous or elastomeric substances such as natural rubber, silicone rubber, and polyisobutylenes. Low molecular weight polyisobutylenes having a viscosity average molecular weight of from about 36,000 to about 58,000 (Florey) possessing pressure sensitive adhesive properties are preferred. Such polyisobutylenes are commercially available under the trademark Vistanex from Exxon as grades LM-MS and LM-MH.

Optionally, one or more thermoplastic elastomers can be included in the pressure sensitive adhesive. These elastomers impart the properties of rubber-like extensibility and both rapid and complete recovery from modular strains to the pressure sensitive adhesive component. Suitable thermoplastic elastomers include medium molecular weight polyisobutylenes having a viscosity average molecular-weight of from about 1,150,000 to U.S. Pat. No. 1,600,000 (Florey), butyl rubber which is a copolymer of isobutylene with a minor amount of isoprene having a viscosity average molecular weight of from about 300,000 to about 450,000 (Florey), and styrene copolymers such as styrene-butadiene-styrene (S-B-S), styrene-isoprene-styrene (S-I-S), and styrene-ethylene/butylene-styrene (S-EB-S) which are commercially available, for example, from Shell Chemical Co. under the trademark Kraton as Kraton D1100, D1102, Kraton D1107, Kraton 4000, Kraton G1600, and Kraton G4600. Preferred thermoplastic elastomers are butyl rubber having a viscosity average molecular weight of about 425,000 (commercially available from Exxon as grades 077 or 065), polyisobutylene having a viscosity average molecular weight of about 1,200,000 (commercially available under the trademark Vistanex from Exxon as grade L-100), and styrene-isoprene-styrene (S-I-S) copolymers (commercially available from Shell as Kraton D1107).

The pressure sensitive adhesive may be present in the from about 20 percent to about 70 percent by weight of the hydrocolloid adhesive composition, preferably from about 25 percent to about 50 percent by weight. The thermoplastic elastomer can be present at from about 20 percent to about three times the weight of the pressure sensitive elastomeric substances.

The water dispersible hydrocolloids provide wet tack for the hydrocolloid adhesive while the pressure sensitive adhesive component provides dry adhesion and imparts structural integrity to the hydrocolloid adhesive.

The hydrocolloid adhesive may also include a plasticizer or solvent such as mineral oil or petrolatum with mineral oil being preferred. The hydrocolloid adhesive may comprise from about 5 percent to about 25 percent by weight of a plasticizer or solvent such as mineral oil or petrolatum with mineral oil being preferred.

The hydrocolloid adhesive may also include a tackifier such as a terpene resin or a pentaerythritol ester of a rosin, such as Pentalyn (RTM) H (from Pinova Inc. of Brunswick, Georgia, USA). The tackifier may be present from about 10 percent to about 25 percent by weight of the hydrocolloid adhesive.

Small amounts, i.e., less than 5 percent by weight of other ingredients may be included within the hydrocolloid adhesive. For example, an antioxidant such as butylated hydroxyanisole, butylated hydroxytoluene, or tetrakis (methylene [3,5-di-tertbutyl-4-hydroxyhydrocinnamate]) methane, Irganox 1010 by BASF.

In one preferred embodiment, the hydrocolloid adhesive comprises 20 to 70 percent by weight of low molecular weight polyisobutylene and said optional thermoplastic elastomer; between about 10 and 65 percent by weight of said hydrocolloids including sodium carbomethylcellulose, pectin and gelatin; between about 5 and 25 percent by weight of a plasticizer or solvent; between about 10 and 25 percent by weight of a tackifier; and up to about 5 percent by weight of antioxidants and/or pharmacologically active ingredients.

In a more preferred embodiment, the hydrocolloid adhesive comprises about 8 percent by weight of said polyisobutylene, about 22 percent by weight of thermoplastic elastomer (preferably about 16 percent butyl rubber and about 8 percent S-I-S rubber), about 11 percent by weight of plasticizer or solvent (preferably mineral oil) about 1 percent by weight of antioxidant (preferably tetrakis (methylene [3,5-di-tertbutyl-4-hydroxyhydrocinnamate]) methane), about 13 percent by weight of tackifier (preferably pentaerythritol ester of a rosin), and about 45 percent by weight of hydrocolloid (preferably about 15 percent gelatin, about 15 percent sodium methylcellulose and about 15 percent pectin).

In some cases, the layer of hydrocolloid adhesive has a thickness of no more than 2 mm. In some cases, the layer of hydrocolloid adhesive has a thickness of no more than 1 mm. In some cases, the layer of hydrocolloid adhesive has a thickness of no more than 0.5 cm.

Providing such a thin layer of hydrocolloid adhesive is advantageous in ensuring that the cannula of the infusion hub enters to the correct depth subcutaneously, as well as having benefits in terms of maintaining a low profile of the infusion hub and economic use of material.

In some cases, the layer of hydrocolloid adhesive has a maximum dimension (e.g. a diameter if circular, or length if not) of no more than 10 cm, for example, no more than 9 cm, or no more than 8 cm. 8 cm is a typical maximum dimension of the adhesive patch of a big patch pump. In some cases the layer of hydrocolloid adhesive has a maximum dimension (e.g. a diameter if circular, or length if not) of no more than 7 cm, no more than 6 cm, no more than 5 cm or no more than 4 cm. 4 cm is a typical maximum dimension (e.g. diameter) of the adhesive patch of a normal infusion set.

In some cases, the layer of hydrocolloid adhesive has a maximum dimension (e.g. a diameter if circular, or length if not) of at least 2 cm, for example at least 3 cm or at least 4 cm (which is a typical maximum dimension, e.g. diameter, of the adhesive patch of a normal infusion set). In some cases the layer of hydrocolloid adhesive has a maximum dimension (e.g. a diameter if circular, or length if not) of at least 5 cm, at least 6 cm, at least 7 cm or at least 8 cm (which is a typical maximum dimension, e.g. diameter, of the adhesive patch of a big patch pump). Certain preferred ranges of maximum dimension include 3 cm-9 cm (which should be sufficient for small infusion sets and big patch pumps); 3 cm-5 cm (which are especially suited to infusion sets); and 6 cm-9 cm (which should accommodate even large patch pumps.

In some cases the surface area of the adhesive patch may be no more than 100 cm², for example no more than 80 cm², or no more than 60 cm². This would be typical for the adhesive patch of a large patch pump of 6 cm×8 cm. In some cases the surface area of the adhesive patch may be no more than 30 cm², for example no more than 20 cm², or no more than 15 cm². This would be typical for the adhesive patch of a normal infusion set with a diameter of 4 cm.

In some cases, the opening is a through-hole. This may be beneficial as a soft cannula (e.g. a polymeric catheter) can be simply threaded through the through-hole; and in some embodiments, the through hole could be arranged around a platform on the skin-facing surface of the infusion hub. Alternative openings, such as slits, or blind holes, may be envisaged, which could be pushed or broken open by the cannula of an infusion hub—this may be more suitable where the cannula is a metal needle.

The hole may have a greatest width, or where circular a diameter of at least 0.3 mm, at least 0.4 mm, at least 0.5 mm, at least 0.6 mm, at least 0.7 mm, at least 0.8 mm, at least 0.9 mm, or at least 1 mm.

The hole may have a greatest width, or where circular a diameter of no more than 2 mm, no more than 1.5 m, no more than 1 mm, no more than 0.9 mm, no more than 0.7 mm, no more than 0.6 mm, or no more than 0.5 mm.

For example, the hole may have a greatest width, or where circular a diameter of between 0.5 mm and 2 mm, for example about 1 mm, or about 1.5 mm. Such a diameter of hole should be sufficient to receive even a cannula of a large diameter for an infusion set, e.g. 25 gauge, but still not have an unduly loose fit around even a cannula of small diameter for an infusion set, e.g. 29 gauge. A relatively close fit of the cannula in the hole can assist in terms of accurate location of the adhesive structure on the infusion hub.

The hole may have a greatest width, or where circular a diameter of at least 3 mm, at least 5 mm, at least 10 mm, at least 15 mm, or at least 20 mm.

The hole may have a greatest width, or where circular a diameter of no more than 50 mm, no more than 30 m, no more than 25 mm, no more than 20 mm, no more than 15 mm, or no more than 10 mm.

For example, the hole may have a greatest width, or where circular a diameter of between 10 mm and 25 mm, for example about 15 mm, or about 20 mm. Such a diameter of hole is sufficient to closely fit around a platform of the hub, from which a cannula extends. A relatively close fit of the platform in the hole can assist in terms good adhesion of the hub to the skin of a patient.

In some cases, the adhesive structure comprises a release liner. The release liner is a releasable temporary covering layer which covers the hydrocolloid adhesive to prevent accidental adhesion prior to use, and is removed by a user to expose the adhesive and adhere the infusion hub to the skin.

The layer of hydrocolloid adhesive may have a skin-facing surface and an opposite infusion hub-facing surface. The release liner may be provided on the skin-facing surface. The release liner may be provided directly on the skin-facing surface of the layer of hydrocolloid adhesive, i.e. there may be no intervening layers between the layer of hydrocolloid adhesive and the release liner.

The layer of hydrocolloid adhesive may have an open cell foam structure. The skin facing surface and the hub facing surfaces may each have closed cells. The layer of hydrocolloid adhesive may have an outer peripheral edge which may also have closed cells. The layer of hydrocolloid adhesive may have an inner peripheral edge (i.e. around the opening) which may also have closed cells. The provision of closed cells at both surfaces and edges may be advantageous in terms of maintaining adhesion.

In some cases, the opening extends through the entire adhesive structure, including the adhesive layer and the release liner. In other cases, the opening may extend through the entire adhesive structure other than the release liner. As the release liner is removed before use, it does not need to include the opening. However, including an opening in the release liner may have benefits, for example in terms of maintaining a non-tacky surface to make attachment of the adhesive structure to the infusion hub easier.

Where the opening extends through both the release liner and the adhesive layer, the opening may be of the same type, or a different type. There are advantages associated with both approaches, for example, a through hole through both layers may be easy to punch out in a single action, whereas a through hole through the adhesive layer and a slit through the release liner may allow for the cannula to be easily inserted through the adhesive layer, whilst the slit in the release liner provides greater coverage of the adhesive surface (i.e. even covering the internal circumferential surface of the hole).

The invention also provides an adaptor for an infusion set a patch pump or a continuous glucose monitor; the adaptor comprising an adhesive structure according to the present disclosure; wherein the layer of adhesive has a skin-facing surface and an opposite infusion hub, patch pump or continuous glucose monitor-facing surface; wherein a release liner is provided on the skin-facing surface; and wherein a protective layer is provided on the opposite surface.

In a second aspect of the invention, there is provided an adaptor for an infusion set; the adaptor comprising an adhesive structure according to the present disclosure; wherein the layer of adhesive has a skin-facing surface and an opposite infusion hub-facing surface; wherein a release liner is provided on the skin-facing surface; and wherein a protective layer is provided on the infusion set-facing surface.

Thus in one embodiment of the invention there is provided an adaptor for an infusion set, the adaptor comprising an adhesive structure comprising a layer of adhesive having at least one opening therethrough for receiving a cannula of the infusion set and a release liner; wherein the layer of adhesive has a skin-facing surface and an opposite infusion hub-facing surface; wherein the release liner is provided on the skin-facing surface; wherein a protective layer is provided on the infusion set-facing surface; and wherein the layer of adhesive is a hydrocolloid adhesive.

The adhesive structure may be the adhesive structure of the first aspect of the invention, and may optionally include any of the optional features set out above.

The protective layer may be adapted to adhere to a further adhesive layer provided on the infusion set.

For example, the protective layer may be adapted to adhere to a further adhesive layer which is an acrylic adhesive or a soft silicone adhesive.

In some cases, the protective layer is not sticky or tacky.

In some cases, the protective layer is irremovable. That is to say, in contrast to the release liner discussed above, the protective layer cannot be simply pulled off the adhesive layer by a user, leaving the adhesive layer intact.

In some cases, the protective layer is a polymer layer. In some cases the protective layer comprises a material selected from the group consisting of nonwoven polyester fabric, cellulose, and polypropylene.

In some cases the protective layer is between about 0.02 mm and about 0.6 mm thick. Such a thin protective layer is advantageous in maintaining a low-profile adaptor.

In some cases, adaptor has a thickness of no more than 2 mm. in some cases, the adaptor has a thickness of no more than 1 mm. In some cases, the adaptor has a thickness of no more than 0.5 cm.

Providing such a thin adaptor is advantageous in ensuring that the cannula of the infusion hub enters to the correct depth subcutaneously, despite the fact that in use the adaptor necessarily provides additional separation between the skin-facing surface of the infusion hub to which it is attached, and the skin, and the length of the cannula of an infusion hub is carefully controlled to extend to the optimum distance from the skin facing surface of the infusion hub.

In some cases the protective layer is a removable release liner, whereby the release liner can be removed and the adaptor can be adhered to a further adhesive layer provided on a hub of the infusion set.

The adaptor has a sandwich structure with a protective backing layer on one side and a release liner on the opposite side.

The invention also provides a method of adapting a (conventional) infusion hub, a patch pump or a continuous glucose monitor to be suitable for use for an extended period of time of more than 7 days, such as an extended period of time of 8-10 days, 10-14 days, and even an extended period of time of 14 or more days; the method comprises providing the adaptor of the second aspect of the invention between the skin-facing surface of the infusion hub, patch pump or continuous glucose monitor and the skin. The method may comprise adhering the infusion hub, patch pump or a continuous glucose monitor to the skin via the layer of adhesive of the adhesive structure.

In a third aspect of the invention there is provided a method of adapting a (conventional) infusion hub, to be suitable for use for an extended period of time of more than 7 days, such as an extended period of time of 8-10 days, 10-14 days, and even an extended period of time of 14 or more days; the method comprises providing the adaptor of the second aspect of the invention between the skin-facing surface of the infusion hub and the skin. The method may comprise adhering the infusion hub to the skin via the layer of adhesive of the adhesive structure.

The method may comprise removing a release liner from the skin-facing surface of the infusion hub to expose a further adhesive layer and adhering the adaptor to the infusion hub via the further adhesive layer.

The method may comprise adhering the further adhesive layer of the infusion hub to the protective layer of the adaptor. Alternatively, where the protective layer of the adaptor is removable, the method may comprise removing the protective layer of the adaptor and adhering the hydrocolloid adhesive layer of the adaptor to the further adhesive layer of the infusion hub.

The further adhesive layer may be an adhesive patch. The further adhesive layer may not be a hydrocolloid adhesive. The further adhesive layer may be an acrylic adhesive, or a soft silicone adhesive.

The method may comprise threading a cannula of the infusion hub through the opening in the adaptor.

The method may comprise aligning the outline of the adaptor with the outline of the infusion hub. The outline of the infusion adaptor may be the same as the outline of the infusion hub, for example, the same as the outline of the adhesive patch of the infusion hub.

The method may comprise adhering the infusion hub to the skin at a single infusion site in use for an extended period of time, for example more than 7 days, 10 or more days, or at least 14 days. As such, the method may comprise leaving the infusion hub adhered to the skin for more than 7 days, e.g. 8-10 days; 10 or more days, e.g. 10-14 days, or at least 14 days, then removing it.

The invention also provides a kit of parts comprising an infusion hub of an infusion set, a patch pump or a continuous glucose monitor and an adaptor according to the disclosure.

According to a fourth aspect of the invention, there is provided a kit of parts comprising an infusion hub of an infusion set and an adaptor according to the second aspect of the invention.

The adaptor may have a corresponding size and shape to the skin-facing side of the infusion hub.

Thus in one embodiment there is provided a kit of parts comprising an infusion hub of an infusion set and an adaptor; the adaptor comprising an adhesive structure comprising a layer of adhesive having at least one opening therethrough for receiving a cannula of an infusion set and a release liner; wherein the layer of adhesive is a hydrocolloid adhesive; wherein the layer of adhesive has a skin-facing surface and an opposite infusion hub-facing surface; wherein the release liner is provided on the skin-facing surface; and wherein a protective layer is provided on the infusion hub-facing surface; the adaptor having a corresponding size and shape to the skin-facing side of the infusion hub.

The adaptor may optionally include any optional features outlined above.

Such a kit may thus be used either with a conventional adhesive layer, which is provided on the hub, or adapted for extended use by attaching the adaptor.

The hub may have a further adhesive layer and the adaptor may have a corresponding size and shape to the further adhesive layer.

In some cases the further adhesive layer is an adhesive patch and the adaptor has a corresponding size and shape to the adhesive patch.

In some cases the further adhesive layer is not a hydrocolloid adhesive. In some cases the further adhesive layer is an acrylic adhesive or a soft silicone adhesive.

The invention also provides an infusion hub of an infusion set, a patch pump or a continuous glucose monitor; the infusion hub, patch pump or continuous glucose monitor comprising an adhesive structure according to the present disclosure.

Thus in one embodiment there is provided an infusion hub of an infusion set, a patch pump or a continuous glucose monitor; the infusion hub, patch pump or continuous glucose monitor comprising an adhesive structure; the adhesive structure comprising a layer of adhesive having at least one opening therethrough for receiving a cannula of the infusion set or patch pump, or a sensor of the continuous glucose monitor and a release liner; wherein the layer of adhesive is a hydrocolloid adhesive.

According to a fifth aspect of the invention, there is provided an infusion hub of an infusion set; the infusion hub comprising an adhesive structure according to the present disclosure. The adhesive structure may be the adhesive structure of the first aspect of the invention. The adhesive structure may include any optional features outlined above.

In some cases, the hub comprises a cannula for insertion into a patient, the cannula extending through the at least one opening through the adhesive structure.

The hub may comprise a housing. The hydrocolloid adhesive may be directly bonded to the underside of the housing with no intervening further adhesive. For example, the hydrocolloid adhesive itself may adhere directly to the underside of the housing, or it may be bonded directly by other means, such as welding, e.g. heat welding or ultrasonic welding. As such, the hydrocolloid adhesive may be a substitute for the known adhesives provided on infusion hubs, which are almost exclusively acrylic adhesives. Alternatively, the hydrocolloid adhesive may be indirectly bonded to the underside of the housing. For example, a layer of a further adhesive may be provided between the hydrocolloid adhesive and the housing, to bond the hydrocolloid adhesive to the housing, or the hydrocolloid adhesive may be provided with a protective backing layer which is bonded to the underside of the housing, e.g. via another adhesive, welding, such as heat welding or ultrasonic welding. This can allow connection between the hydrocolloid adhesive and the housing, even if the housing is formed from a material to which a hydrocolloid adhesive cannot adequately adhere to.

The hub may comprise a fluid transfer part. The fluid transfer part may be in fluid communication with the cannula. The fluid transfer part may be connected to the housing. The fluid transfer part may be configured to engage with a tubing connector. The cannula may define a downstream end of a fluid flow path extending between a pump and the patient via the fluid transfer part.

The infusion hub may be for subcutaneous infusion of a therapeutic agent into a patient.

In some cases, there is provided an infusion set comprising the infusion hub and a tubing connector configured to engage the fluid transfer part and connectable to tubing for receiving a therapeutic agent from a pump.

In some cases the infusion set comprises the tubing which is connectable between the tubing connector and a pump.

In some cases the infusion set comprises a pump for containing and delivering the therapeutic agent to the infusion hub via the tubing and the tubing connector.

In some cases, an interface of the fluid transfer part comprises a sealing membrane configured to prevent egress of fluid out of the fluid transfer part from the fluid flow path.

In some cases, when the tubing connector is secured to the fluid transfer part, a needle of the tubing connector pierces the sealing membrane of the fluid transfer part.

Thus in one embodiment there is provided an infusion set comprising the infusion hub of the fifth aspect of the invention; a tubing connector configured to engage the fluid transfer part and connectable to tubing for receiving a therapeutic agent from a pump; the tubing which is connectable between the tubing connector and a pump; and a pump for containing and delivering the therapeutic agent to the infusion hub via the tubing and the tubing connector; wherein an interface of the fluid transfer part comprises a sealing membrane configured to prevent egress of fluid out of the fluid transfer part from the fluid flow path; and when the tubing connector is secured to the fluid transfer part, a needle of the tubing connector pierces the sealing membrane of the fluid transfer part.

In some cases, the therapeutic agent comprises insulin, heparin, apomorphine, arbidopa, or levodopa and/or levodopa products.

In some cases, the cannula may be a soft polymeric catheter or a metal needle. The cannula may comprise one of a polytetrafluoroethylene (PTFE), a fluorinated ethylene propylene (FEP), a rubber, a polyethylene (PE), a polyurethane (PU), a polypropylene (PP) or a silicone material. The cannula may be insertable in the patient by an insertion needle.

The invention also provides a method of attaching an infusion set, a patch pump or a continuous glucose monitor; to the skin, comprising: providing the adhesive structure according to the disclosure on a skin-facing surface of an infusion hub of an infusion set, a patch pump or a continuous glucose monitor, and adhering the infusion hub, patch pump or continuous glucose monitor to the skin via the layer of adhesive of the adhesive structure.

According to a sixth aspect of the invention there is provided a method of attaching an infusion set to the skin, comprising: providing the adhesive structure according to the disclosure on a skin-facing surface of an infusion hub of an infusion set, and adhering the infusion hub to the skin via the layer of adhesive of the adhesive structure.

Thus, one embodiment of the invention provides a method of adapting an infusion hub, to be suitable for use for an extended period of time of more than 7 days; the method comprising providing an adaptor between the skin-facing surface of the infusion hub and skin; the adaptor comprising an adhesive structure comprising a layer of adhesive having at least one opening therethrough for receiving a cannula of an infusion set and a release liner; wherein the layer of adhesive is a hydrocolloid adhesive wherein the layer of adhesive has a skin-facing surface and an opposite infusion hub-facing surface; wherein the release liner is provided on the skin-facing surface; and wherein a protective layer is provided on the infusion hub-facing surface; and the method comprising adhering the infusion hub to the skin via the layer of adhesive of the adhesive structure.

The adhesive structure may be the adhesive structure of the first aspect of the invention. The adhesive structure may include any optional features described above.

The infusion hub may be the infusion hub of the fifth aspect of the invention (and may include any of the optional features described above).

The method may comprise adhering the infusion hub to the skin at a single infusion site in use for an extended period of time, for example more than 7 days, 10 or more days, or at least 14 days. As such, the method may comprise leaving the infusion hub adhered to the skin for more than 7 days, 10 or more days, or at least 14 days, then removing it.

The invention also provides a method of forming an adhesive structure according to the present disclosure comprising providing a layer of hydrocolloid adhesive; providing a release liner and providing a hole in the layer of hydrocolloid adhesive for receiving a cannula of an infusion set or a patch pump, or a sensor of a continuous glucose monitor.

According to a seventh aspect of the invention there is provided a method of forming an adhesive structure according to the present disclosure comprising providing a layer of hydrocolloid adhesive; providing a release liner and providing a hole in the layer of hydrocolloid adhesive for receiving a cannula of an infusion set.

The method may comprise extruding the layer of hydrocolloid adhesive. For example, the method may comprise melting a mass of hydrocolloid adhesive and extruding the layer of hydrocolloid adhesive through a die, such as a letter box die. The method may comprise laminating the hydrocolloid adhesive to the release liner and optionally to the optional protective layer to form a laminate. The method may comprise cutting the extruded laminate, for example cutting the laminate with a die tool, e.g. cutting the laminate such that its outline matches the outline of an adhesive patch for an infusion hub. The die tool may be configured to provide the hole. As such, the die tool may comprise a punch.

The die tool may be heated. For example, the die tool may be heated to at least 50 degrees C., for example at least 100 degrees C. Those skilled in the art will easily determine a suitable temperature for heating, in order to provide a clean cut and close any open cells where the adhesive layer is an open-celled structure.

The method of providing a hole may be punching a hole. The hole may be punched with a heated die or punch.

The method may be a method of forming an adhesive structure according to the first aspect of the invention (optionally including any optional features).

Thus in one embodiment there is provided a method of forming an adhesive structure comprising a layer of adhesive having at least one opening therethrough for receiving a cannula of an infusion set and a release liner; wherein the layer of adhesive is a hydrocolloid adhesive; the method of forming the adhesive structure comprising: providing a layer of hydrocolloid adhesive; providing a release liner and providing a hole in the layer of hydrocolloid adhesive for receiving a cannula of an infusion set; wherein the method of providing a hole is punching a hole with a heated die or punch; the method comprising providing a protective layer which is irremovable from the hydrocolloid adhesive layer and is provided on the opposite side of the layer of hydrocolloid adhesive to the release liner; and providing a hole in the layer of hydrocolloid adhesive and in the protective layer in the same action by punching a hole through both layers at the same time.

The method may be a method of forming an adaptor according to the second or fourth aspects of the invention (optionally including any optional features).

The method may be a method of forming an infusion hub according to the fifth aspect of the invention (optionally including any optional features).

The method may comprise providing the release liner on the layer of hydrocolloid adhesive and then providing (e.g. punching) a hole in the layer of hydrocolloid adhesive. The method may comprise providing a hole in both the layer of hydrocolloid adhesive and the release liner in the same action (e.g. punching a hole through both layers at the same time).

The method may comprise providing (e.g. punching) a hole in the hydrocolloid layer, then providing a release liner on the hydrocolloid layer.

The method may comprise providing a protective layer (which may be irremovable from the hydrocolloid adhesive layer and may be provided on the opposite side of the layer of hydrocolloid adhesive to the release liner). The method may comprise providing a hole in the layer of hydrocolloid adhesive and in the protective layer in the same action (e.g. punching a hole through both layers at the same time).

The method may comprise providing (e.g. punching) a hole in the hydrocolloid layer and the protective layer, then providing a release liner on the hydrocolloid layer.

The method may comprise providing a hole in the protective layer, the layer of hydrocolloid adhesive and the release liner in the same action (e.g. punching a hole through all three layers at the same time).

The method may further comprise packaging the adhesive structure having the hole therein and sterilising the packaged adhesive structure.

The method may comprise forming an adaptor comprising the adhesive structure then packaging the adaptor. The method may comprise sterilising the adaptor. The method may comprise first packaging the adaptor, then sterilising the adaptor in the package. The method may comprise sterilising the adaptor then packaging it (in a sterile environment).

The method may comprise forming an infusion hub comprising the adhesive structure then packaging the infusion hub. The method may comprise sterilising the infusion hub. The method may comprise first packaging the infusion hub, then sterilising the infusion hub in the package. The method may comprise sterilising the infusion hub then packaging it (in a sterile environment).

The method may comprise attaching the layer of hydrocolloid adhesive to the skin-facing side of the housing of an infusion hub. The method may comprise attaching one side of the layer of hydrocolloid adhesive to the skin-facing side of the housing of an infusion hub and releasably attaching the opposite side to the release liner.

### BRIEF DESCRIPTION OF THE DRAWINGS

Exemplary infusion sets and adhesive adaptors for infusion sets are further described hereinafter with reference to the accompanying drawings, in which:

FIG. 1A is an illustration of an infusion set in situ;

FIG. 1B is a further illustration of the infusion set of FIG. 1A;

FIG. 1C is a further illustration of the infusion set of FIG. 1A prior to use; and

FIG. 1D is an enlarged illustration of a portion of the infusion set of FIG. 1C in the region of the adhesive patch;

FIG. 2A is a plan view of an exemplary adhesive adaptor;

FIG. 2B is a side cross-section view of the adhesive adaptor of FIG. 2A;

FIG. 2C is a side cross-section view of the adhesive adaptor of FIG. 2A applied to an infusion hub; and

FIG. 2D is a side cross section view of the adhesive adaptor of FIG. 2A applied to an infusion hub and arranged in-situ at an infusion site.

### DETAILED DESCRIPTION

The presently described infusion sets have particular application for use with infusion pump systems such as an infusion pump for delivery of a therapeutic agent, such as insulin, heparin or any other liquid therapeutic agents, where the infusion pump includes a fluid pump and a reservoir, and an infusion set having a cannula (typically part of an infusion hub) and tubing for connecting the cannula to the reservoir. The infusion pump may be an insulin pump for CSII therapy, and the therapeutic agent may be an insulin formulation. The presently described infusion sets are able to deliver insulin to a patient at a single infusion site over an extended period of time. An extended period of time is to be understood to mean more than 7 days, i.e. at least eight days. More specifically, an extended period of time may include 8 to 10 days, 10 to 14 days, or 14 or more days.

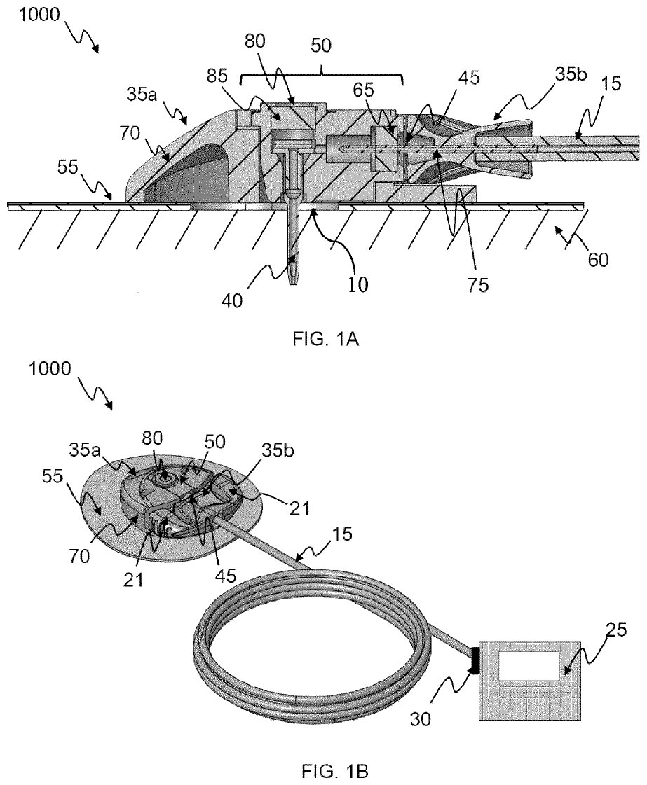

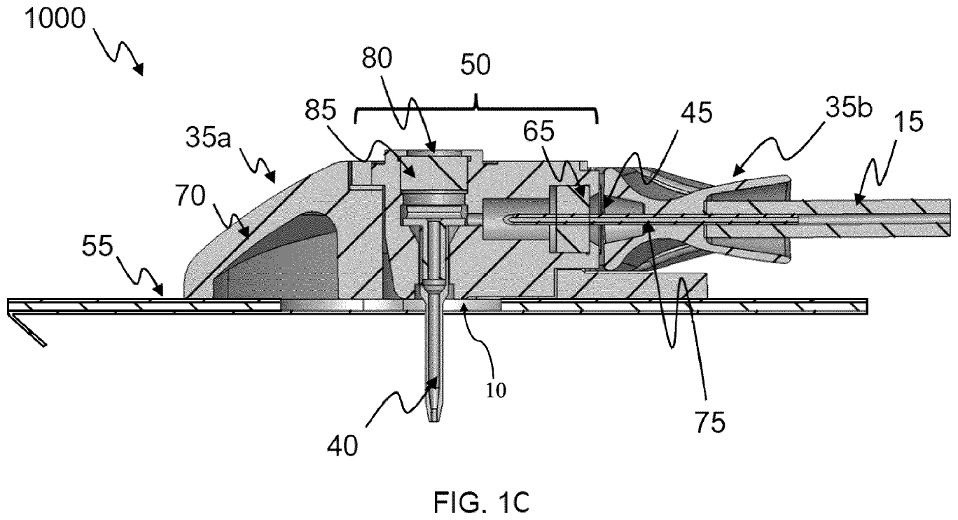

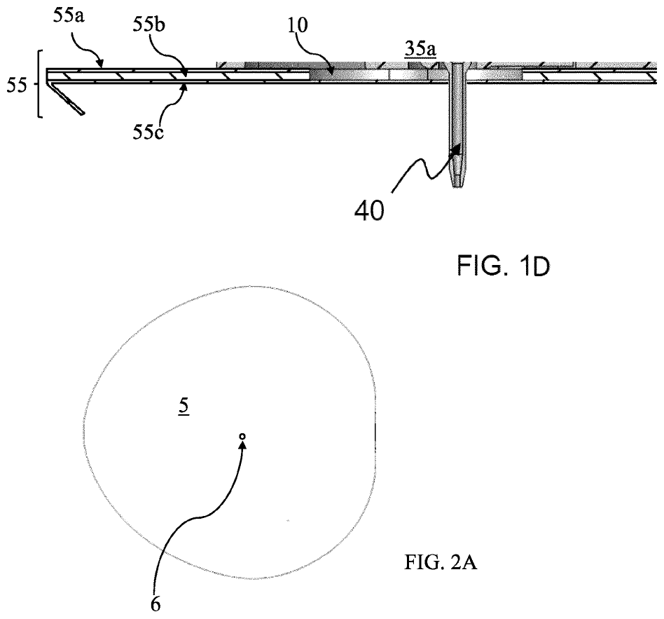

FIGS. 1A and 1B illustrate an infusion set 1000 on an infusion site. The infusion set 1000 includes an infusion hub 35a and a tubing connector 35b. The infusion hub 35 is secured to the skin of a patient by an adhesive structure, in the form of an adhesive patch 55 which maintains a cannula 40 subcutaneously within the sub-dermal fatty tissue of a patient. Tubing 15 connects a pump outlet connector 30 of a pump 25 (the pump 25 containing the therapeutic agent) with the infusion hub 35a via the tubing connector 35b. The infusion hub 35a includes a housing 70, and a fluid transfer part 50 which provides a fluid channel. The fluid transfer part is secured within the housing 70 and retains the cannula 40 within the patient. The fluid part 50 may be integral with the housing 70.

The tubing connector 35b engages an interface 45 of the fluid transfer part 50 to connect a downstream end of the tubing 15 to the fluid transfer part 50. The tubing connector 35b includes a releasable connector, in this case a releasable snap-fit joint. As shown in FIG. 1B, the tubing connector 35b has mechanical elements in the form of resiliently deformable arms 21 that engage corresponding mechanical receiving elements on the hub 35a, or more specifically, the housing 70 of the infusion hub 35a. While the mechanical elements are arranged as a releasable clip in the illustrated tubing connector 35b, it would be apparent that this is not essential. When the tubing connector 35b is secured to the infusion hub 35a, a needle 75 of the tubing connector 35b pierces a sealing membrane 65 within the fluid transfer part 50, so that the therapeutic agent can flow through the fluid transfer part 50 and into the patient via the cannula 40.

The function of the fluid transfer part 50 is to allow fluid (e.g. a therapeutic agent) to be transferred through the infusion hub 35a (i.e., from the interface 45 of the fluid transfer part 50 to the cannula 40). As can be seen in FIG. 1A, the fluid transfer part 50 provides a fluid flow path that extends firstly approximately parallel to the plane of the skin surface, and then bends to be approximately perpendicular to the skin surface.

The fluid transfer part 50 has multiple interfaces 45, 80. A sealing membrane 65 seals a first interface 45, and a second sealing membrane 85 is used to seal a second interface 80. While two interfaces 45, 80 are shown, it would be apparent more than two interfaces may be provided as required. Some or all the interfaces may have a sealing membrane secured therein to prevent egress of therapeutic agent from the fluid transfer part 50, through the respective interface.

The cannula 40 is a substantially tubular member for insertion in, and delivering a therapeutic agent to, an infusion site 60. The therapeutic agent of this embodiment includes insulin or an insulin solution. A proximal end of the cannula 40 is fluidly connected to a source of a therapeutic agent, here an infusion pump 25. An opposing, distal end of the cannula 40 is positioned in the infusion site 60, extending to a desired depth to deliver the therapeutic agent. The cannula 40 is any suitable cannula suitable for implantation in a tissue site of a patient, such as a polymeric catheter or metal needle.

FIGS. 1C and 1D illustrate the infusion set of FIGS. 1A and 1B prior to use. As best seen in FIG. 1D, the adhesive patch 55 is formed of a layer of hydrocolloid adhesive 55b, which is sandwiched between a protective layer 55a and a release liner 55c.

The protective layer 55a, which in this illustrative example is a polymer layer, is irremovably bonded (e.g. welded) to the skin-facing surface of the housing 70 of the infusion hub 35a. In this example, a platform 10 is located around the cannula 40, providing a flat base region through which the cannula extends. The platform 10 has a depth corresponding to the combined depth of the protective layer 55a and the hydrocolloid adhesive 55b, for example about 0.5 to 1 mm, and sits snugly within an opening in the protective layer 55a and hydrocolloid adhesive 55b of the adhesive patch 55. In this illustrative embodiment, the diameter of the opening and of the platform 10 is about 15 mm.

On the opposite side to the protective layer 55a, i.e. on the skin-facing side of the layer of hydrocolloid adhesive, the removeable release liner 55c is provided. As can be seen, the release liner comprises an optional release tab to aid its removal, and the release liner extends across the entire skin-facing surface of the layer of hydrocolloid adhesive 55b, also covering the platform 10, but with the cannula 40 extending through a through-hole in the release liner 55c.

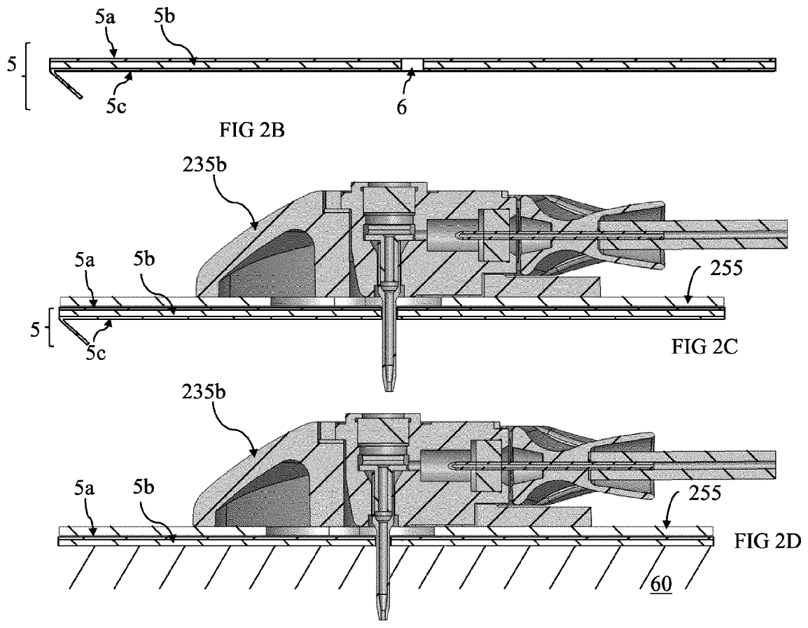

FIGS. 2A and 2B illustrate an exemplary adaptor 5 for adapting the infusion hub of an infusion set for use for an extended period of time. The purpose of such an adaptor is to be an interface between an infusion hub having an adhesive that is unsuitable for extended wear times (e.g. an acrylic adhesive) and the skin of the patient. As such, the outline of the adaptor corresponds to the shape of the adhesive patch of the infusion hub for which the adaptor is intended to be used. In this exemplary embodiment, the outline of the adaptor 5 is generally approximate to a circular triangle, but the precise shape is not important, only that it matches the outline of the adhesive patch of the infusion hub for which it is intended to be used.

Much like the adhesive patch 55 discussed in relation to the first embodiment, the adaptor 5 is formed of a layer of hydrocolloid adhesive 5b, which is sandwiched between a protective layer 5a and a release liner 5c.

In this exemplary embodiment, the protective layer is between about 0.02 mm and about 0.6 mm thick, e.g. 0.3 mm thick, whilst the hydrocolloid adhesive is about 0.7 mm thick, and the release liner is about 0.1 mm thick. As such the adaptor has a thickness of only slightly more than 1 mm.

The protective layer 5a, which in this illustrative example is a polymer layer, is intended to be attached, in use, to a further adhesive layer of the adhesive patch of the infusion hub which the adaptor is intended to adapt.

On the opposite side to the protective layer 5a, i.e. on the skin-facing side of the layer of hydrocolloid adhesive 5b, the removeable release liner 5c is provided. As can be seen, the release liner 5c comprises an optional release tab to aid its removal, and the release liner 5c extends across the entire skin-facing surface of the layer of hydrocolloid adhesive 5b.

In this embodiment, a through-hole 6 has been punched through all three layers 5a, 5b, 5c. The through hole 6 of this particular embodiment has a diameter of just 1 mm, which is sufficient to accommodate the cannula of the infusion hub, with which the adaptor is intended to operate. In other embodiments, not illustrated, the through-hole may be provided only through the protective layer and the hydrocolloid layer; no opening, or a different type of opening, e.g. a slit, may be provided in the release liner.

The adaptor of FIGS. 2A to 2B may be supplied as a kit together with an infusion hub, and FIG. 2C shows the infusion hub 235 of such a kit adhered, via the further adhesive of its own adhesive patch 255 to the protective layer 5a of the adaptor 5. As can be seen from FIG. 2C, the cannula 240 of the infusion hub 235 extends through the through-hole 6 in the adaptor. Of course, to attach the infusion hub 235 to the skin, the release liner 5c is removed, and as shown in FIG. 2D, the infusion hub is adhered to the skin via the hydrocolloid adhesive layer 5b.

The hydrocolloid adhesive layer 55b of the infusion hub 35b and the hydrocolloid adhesive layer 5b of the adaptor 5 may comprise a homogeneous blend of one or more pressure sensitive adhesive materials and one or more water dispersible hydrocolloid materials. The hydrocolloid adhesive layers may also include a tackifier and a plasticizer or solvent. Additionally one or more thermoplastic elastomers may be included with the pressure sensitive adhesive material, as well as, one or more natural or synthetic polymers capable of developing elastomeric properties when hydrated.

### Example 1

An adhesive adaptor was provided with a hydrocolloid adhesive layer having the following composition:

| | PERCENTAGE |
|---|---|
| Ingredients | BY WEIGHT |
| GELATIN | 15 |
| SODIUM | 15 |
| CARBOXYMETHYLCELLULOSE | |
| PECTIN | 15 |
| POLYISOBUTYLENE LMMH | 8 |
| BUTYL RUBBER | 16.25 |
| STYRENE-ISOPRENE-STYRENE | 6 |
| RUBBER (Kraton 1107) | |
| TETRAKIS (METHYLENE [3,5- | 0.5 |
| DI-TERTBUTYL-4- | |
| HYDROXYHYDROCINNAMATE]) | |
| METHANE (Irganox 1010) | |
| PENTAERYTHRITOL ESTER OF | 12.75 |
| A ROSIN (Pentalyn H) | |
| MINERAL OIL | 11.5 |

The mineral oil, polyisobutylene, butyl rubber, Kraton 1107, and Irganox 1010 were combined in a sigma blade mixer with heating (to about 115 DEG C.) and agitating for approximately 1.5 hours. The mixture was cooled to about 100 DEG C. and after another 30 minutes of blending, the gelatin, sodium carboxymethylcellulose, pectin and Pentalyn H were added. Mixing was continued at about 100 DEG C. for 30 minutes until a homogeneous mass was obtained.

This mass was allowed to cool and was then flattened to a thickness of about 0.05 cm. Silicon release paper was applied to both sides.

One piece of release paper was removed from a strip of the so-formed hydrocolloid adhesive. A 0.3 mm thick protective backing layer of polymer film was placed over the exposed surface of the strip. This three layer structure was placed into an oven and heated at about 110 DEG C. for about 5 minutes. The structure was then pressed through rollers with about a 1 mm spacing. This rendered the polymer film irremovably stuck to one side of the hydrocolloid adhesive layer, whilst the release liner could be removed without damaging the hydrocolloid adhesive.

The resulting adhesive structure was cut into the desired shape with a heated die, had a hole 6 punched through with a heated punch, was packaged and sterilized.

Following the procedure of Example 1, but employing the following ingredients on a weight percentage basis, other adhesive structures within the scope of the invention are obtained.

| | EXAMPLE | | | | | | | | | | | |
|---|---|---|---|---|---|---|---|---|---|---|---|---|
| Ingredient | 2 | 3 | 4 | 5 | 6 | 7 | 8 | 9 | 10 | 11 | 12 | 13 |
| Styrene-isoprene-styrene | 20.53 | — | — | — | 6.75 | 20 | 6 | — | — | — | 6.75 | 10 |
| copolymer (Kraton 1107) | | | | | | | | | | | | |
| Polyisobutylene | 9.62 | 40 | 20 | 15 | 8 | 10 | 8 | 40 | 20 | 15 | 8 | 20 |
| (Vistanex LM-MH) | | | | | | | | | | | | |
| Polyisobutylene | — | — | 25 | — | — | — | — | — | 25 | — | — | — |
| (Vistanex 100) | | | | | | | | | | | | |
| Sodium Carboxymethyl- | — | 20 | 35 | 15 | 14.34 | 15 | 10 | — | — | — | — | — |
| cellulose | | | | | | | | | | | | |
| Guar Gum | — | — | 20 | 20 | — | 20 | — | — | — | — | — | — |
| Butyl Rubber (Grade 065) | — | — | — | — | 16.25 | — | 16.25 | — | — | — | 16.25 | — |
| Antioxidant (Irganox 1010) | 1.54 | — | — | 0.5 | 0.5 | — | 0.5 | — | — | 0.5 | 0.5 | — |
| Pectin | — | 20 | — | — | 14.33 | — | — | — | — | — | — | — |
| Gelatin | — | 20 | — | — | 14.33 | — | — | — | — | — | — | — |
| Mineral Oil | 20.44 | — | — | 9.5 | 13.5 | 20 | 11.5 | — | — | 9.5 | 13.5 | 20 |
| Tackifier (Pentalyn H) | 12.84 | — | — | 20 | 12 | 15 | 12.75 | — | — | 20 | 12 | 15 |
| Butyl Rubber (Grade 077) | — | — | — | 20 | — | — | — | — | — | 20 | — | — |
| Sodium Alginate (Kelset | 35.03 | — | — | — | — | — | 35 | 60 | 55 | 35 | 45 | 35 |
| Powder) | | | | | | | | | | | | |

The adaptors may be packaged together with infusion hubs comprising an adhesive patch with a skin-facing adhesive other than a hydrocolloid adhesive (e.g. an acrylic adhesive) as is conventional, as a kit, in which the conventional adhesive may be used for ordinary short-term attachment at the infusion site, or the adaptor may be introduced between the conventional adhesive and the skin at the infusion site, to adapt the infusion hub for extended wear times.

### Examples 14-26

In example 14, infusion hubs as described in relation to FIGS. 1A-D are obtained by following the procedure of Example 1 up to the point of cutting the adhesive structure to desired shape. Then, a larger hole, corresponding in size and position to the platform 10 is punched in the adhesive structure, and the upper side of the polymer film of the structure, is adhered to the underside of the housing 70 to form the adhesive patch 55.

In examples 15-26, the procedure of example 14 is followed, but using an adhesive structure formed in accordance with examples 2-13 respectively, to obtain infusion hubs comprising an adhesive structure with a layer of hydrocolloid adhesive.

In industrial production, the adhesive structure can be manufactured using an ELC (Extrusion, Lamination and Cutting) machine, whereby the mass of hydrocolloid adhesive is melted, extruded through a letterbox die to the required thickness, laminated to the protective backing film and the release liner, then cut out to the desired dressing shape, with the hole 6 punched through via a heated die tool. This technique allows a clean cut while closing any exposed open cells at the inner/outer peripheral edges and can be easily implemented into a production line.

Of course, those skilled in the art will be able to use routine methods to select a suitable ELC machine and adjust parameters such as the heat of the dye-cutter and the material of the cutting edge to achieve the desired result, in which all cells of both the infusion-hub facing surface and the skin facing surface, as well as the inner and outer peripheral edges of the layer of hydrocolloid adhesive are closed.

In use, the infusion hubs 35a of the invention are used in just the same way as a conventional infusion hub, i.e. the release liner 55c of the adhesive patch 55 is removed, the hub 35a is attached to an infusion site, and connected, via the tubing connector 35b to a suitable pump. The difference in terms of use is simply that the hub may be left in place for an extended period of time, i.e. more than 7 days, such as 10-14 or more than 14 days.

Use of the adaptors of the invention is also very similar to use of a conventional hub. In the first step, the release liner of the adhesive patch of a conventional hub is removed. Then, rather than sticking the conventional hub to the skin, the skin-facing side of the adhesive patch of the conventional hub is instead arranged facing the protective backing layer 5a of the adhesive adaptor 5. The cannula of the hub is introduced through the opening 6 in the adhesive adaptor 5, and the adaptor 5 is carefully stuck to the hub, with the matching outlines of the adhesive patch and the adaptor being aligned. Then, to adhere the hub to the skin at the infusion site, the release liner 5c of the adaptor 5 is removed, and the hub is attached to the skin in the conventional manner (for example using an insertion device), adhered to the skin by the hydrocolloid adhesive layer 5b, such that it can remain in place for an extended period of time, i.e. more than 7 days, such as 10-14 days, or more than 14 days.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The invention is not restricted to the details of any foregoing embodiments. For example, it may apply equally to patch pumps or continuous glucose monitors. The invention extends to any novel one, or any novel combination, of the features disclosed in this specification (including any accompanying claims, abstract and drawings), or to any novel one, or any novel combination, of the steps of any method or process so disclosed.

1. A method of adapting an infusion hub, to be suitable for use for an extended period of time of more than 7 days;
the method comprising providing an adaptor between the skin-facing surface of the infusion hub and skin;
the adaptor comprising an adhesive structure comprising a layer of adhesive having at least one opening therethrough for receiving a cannula of an infusion set and a release liner;
wherein the layer of adhesive is a hydrocolloid adhesive wherein the layer of adhesive has a skin-facing surface and an opposite infusion hub-facing surface;
wherein the release liner is provided on the skin-facing surface;
and wherein a protective layer is provided on the infusion hub-facing surface;
and the method comprising adhering the infusion hub to the skin via the layer of adhesive of the adhesive structure.

2. A method of adapting an infusion hub, to be suitable for use for an extended period of time of 8-10 days comprising the steps of claim 1.

3. A method of adapting an infusion hub, to be suitable for use for an extended period of time of 10-14 days comprising the steps of claim 1.

4. A method of adapting an infusion hub, to be suitable for use for an extended period of time of 14 or more days comprising the steps of claim 1.

5. A kit of parts comprising an infusion hub of an infusion set and an adaptor;
the adaptor comprising an adhesive structure comprising a layer of adhesive having at least one opening therethrough for receiving a cannula of an infusion set and a release liner;
wherein the layer of adhesive is a hydrocolloid adhesive;
wherein the layer of adhesive has a skin-facing surface and an opposite infusion hub-facing surface;
wherein the release liner is provided on the skin-facing surface;
and wherein a protective layer is provided on the infusion hub-facing surface;
the adaptor having a corresponding size and shape to the skin-facing side of the infusion hub.

6. An infusion hub of an infusion set, a patch pump or a continuous glucose monitor;
the infusion hub, patch pump or continuous glucose monitor comprising an adhesive structure;
the adhesive structure comprising a layer of adhesive having at least one opening therethrough for receiving a cannula of the infusion set or patch pump, or a sensor of the continuous glucose monitor, and a release liner; wherein the layer of adhesive is a hydrocolloid adhesive.

7. An infusion hub according to claim 6; the infusion hub being for subcutaneous infusion of a therapeutic agent into a patient; comprising a housing and a cannula for insertion into a patient, the cannula extending through the at least one opening through the adhesive structure and being in fluid communication with a fluid transfer part;
the fluid transfer part configured to engage with a tubing connector.

8. An infusion hub according to claim 7 wherein the hydrocolloid adhesive is indirectly adhered to the underside of the housing, via another adhesive.

9. An infusion set comprising the infusion hub of either of claim 7 or 8; a tubing connector configured to engage the fluid transfer part and connectable to tubing for receiving a therapeutic agent from a pump; the tubing which is connectable between the tubing connector and a pump; and a pump for containing and delivering the therapeutic agent to the infusion hub via the tubing and the tubing connector; wherein an interface of the fluid transfer part comprises a sealing membrane configured to prevent egress of fluid out of the fluid transfer part from the fluid flow path; and when the tubing connector is secured to the fluid transfer part, a needle of the tubing connector pierces the sealing membrane of the fluid transfer part.

10. An adaptor for an infusion set, the adaptor comprising an adhesive structure comprising a layer of adhesive having at least one opening therethrough for receiving a cannula of the infusion set and a release liner;
wherein the layer of adhesive has a skin-facing surface and an opposite infusion hub-facing surface;
wherein the release liner is provided on the skin-facing surface;
wherein a protective layer is provided on the infusion set-facing surface;
and wherein the layer of adhesive is a hydrocolloid adhesive.

11. An adaptor according to claim 10 wherein the protective layer is a polymer layer; and is irremovable from the layer of hydrocolloid adhesive.

12. An adaptor according to claim 10 or 11 wherein the protective layer is not sticky or tacky and is adapted to adhere to a further adhesive layer provided on the infusion set, wherein the further adhesive layer is an acrylic adhesive or a soft silicone adhesive.

13. An adaptor according to any of claims 10 to 12 wherein the adaptor has a thickness of no more than 1 mm.

14. A method of forming an adhesive structure comprising a layer of adhesive having at least one opening therethrough for receiving a cannula of an infusion set and a release liner;
wherein the layer of adhesive is a hydrocolloid adhesive;
the method of forming the adhesive structure comprising:
providing a layer of hydrocolloid adhesive;
providing a release liner and providing a hole in the layer of hydrocolloid adhesive for receiving a cannula of an infusion set;
wherein the method of providing a hole is punching a hole with a heated die or punch;
the method comprising providing a protective layer which is irremovable from the hydrocolloid adhesive layer and is provided on the opposite side of the layer of hydrocolloid adhesive to the release liner;
and providing a hole in the layer of hydrocolloid adhesive and in the protective layer in the same action by punching a hole through both layers at the same time.

15. A method of forming an adhesive structure according to claim 14 further comprising packaging the adhesive structure having the hole therein and sterilising the packaged adhesive structure.

16. A method of adapting an infusion hub, to be suitable for use for an extended period of time of more than 7 days according to any one of claims 1 to 4; a kit of parts according to claim 5; an infusion hub or a patch pump according to any one of claims 6 to 8; an infusion set according to claim 9; an adaptor according to any one of claims 10 to 13; or a method of forming an adhesive structure according to either of claim 14 or 15 wherein the hydrocolloid adhesive is a blend of one or more pressure sensitive adhesives and one or more water dispersible hydrocolloids.

17. A method of adapting an infusion hub, to be suitable for use for an extended period of time of more than 7 days according to claim 16; a kit of parts according to claim 16; an infusion hub or a patch pump according to claim 16; an infusion set according to claim 16; an adaptor according to claim 16; or a method of forming an adhesive structure according to claim 16 wherein the one or more water dispersible hydrocolloids are selected from the group consisting of sodium carbomethylcellulose, pectin and gelatin.

18. A method of adapting an infusion hub, to be suitable for use for an extended period of time of more than 7 days according to any one of claim 1 to 4, 16 or 17; a kit of parts according to claim 5, 16 or 17; an infusion hub or a patch pump according to any one of claim 6 to 8, 16 or 17; an infusion set according to claim 9, 16 or 17; an adaptor according to any one of claim 10 to 13, 16 or 17; or a method of forming an adhesive structure according to any one of claims 14 to 17 wherein the hydrocolloid adhesive comprises 20 to 70 percent by weight of low molecular weight polyisobutylene and optional thermoplastic elastomer; between about 10 and 65 percent by weight of hydrocolloids including sodium carbomethylcellulose, pectin and gelatin; between about 5 and 25 percent by weight of a plasticizer or solvent; between about 10 and 25 percent by weight of a tackifier; and up to about 5 percent by weight of antioxidants and/or pharmacologically active ingredients.

19. A method of adapting an infusion hub, to be suitable for use for an extended period of time of more than 7 days according to any one of claims 1 to 4, or 16 to 18; a kit of parts according to claim 5, or any one of claims 16 to 18; an infusion hub or a patch pump according to any one of claims 6 to 8, or 16 to 18; an infusion set according to any one of claims 9 or 16 to 18; an adaptor according to any one of claims 10 to 13, or 16 to 18; or a method of forming an adhesive structure according to any one of claims 14 to 18 wherein the hydrocolloid adhesive comprises about 8 percent by weight of said low molecular weight polyisobutylene, about 16 percent butyl rubber, about 8 percent S-I-S rubber, about 11 percent by weight of mineral oil, about 1 percent by weight of tetrakis (methylene [3,5-di-tertbutyl-4-hydroxyhydrocinnamate]) methane, about 13 percent by weight of pentaerythritol ester of a rosin, about 15 percent gelatin, about 15 percent sodium methylcellulose and about 15 percent pectin.

20. A method of adapting an infusion hub, to be suitable for use for an extended period of time of more than 7 days according to any one of claims 1 to 4, or 16 to 19; a kit of parts according to claim 5, or any one of claims 16 to 19; an infusion hub or a patch pump according to any one of claims 6 to 8, or 16 to 19; an infusion set according to any one of claims 9 or 16 to 19; an adaptor according to any one of claims 10 to 13, or 16 to 19; or a method of forming an adhesive structure according to any one of claims 14 to 19 wherein the layer of hydrocolloid adhesive has a thickness of no more than 1 mm.

21. A method of adapting an infusion hub, to be suitable for use for an extended period of time of more than 7 days according to any one of claims 1 to 4, or 16 to 20; a kit of parts according to claim 5, or any one of claims 16 to 20; an infusion hub or a patch pump according to any one of claims 6 to 8, or 16 to 20; an infusion set according to any one of claims 9 or 16 to 20; an adaptor according to any one of claims 10 to 13, or 16 to 20; or a method of forming an adhesive structure according to any one of claims 14 to 20 wherein the opening is a through-hole.

22. A method of adapting an infusion hub, to be suitable for use for an extended period of time of more than 7 days according to claim 21; a kit of parts according to claim 21; an infusion hub or a patch pump according to claim 21; an infusion set according to claim 21; an adaptor according to claim 21; or a method of forming an adhesive structure according to claim 21 wherein the hole has a greatest width, or where circular a diameter of between 0.5 mm and 2 mm.

23. A method of adapting an infusion hub, to be suitable for use for an extended period of time of more than 7 days according to claim 21; a kit of parts according to claim 21; an infusion hub or a patch pump according to claim 21; an infusion set according to claim 21; an adaptor according to claim 21; or a method of forming an adhesive structure according to claim 21 wherein the hole has a greatest width, or where circular a diameter of between 10 mm and 25 mm.

24. A method of adapting an infusion hub, to be suitable for use for an extended period of time of more than 7 days according to any one of claims 1 to 4, or 16 to 22; a kit of parts according to claim 5, or any one of claims 16 to 22; an infusion hub or a patch pump according to any one of claims 6 to 8, or 16 to 22 wherein the layer of hydrocolloid adhesive has a skin-facing surface and an opposite infusion hub-facing surface; an infusion set according to any one of claims 9 or 16 to 22 wherein the layer of hydrocolloid adhesive has a skin-facing surface and an opposite infusion hub-facing surface; an adaptor according to any one of claims 10 to 13, or 16 to 22; or a method of forming an adhesive structure according to any one of claims 14 to 22 wherein the layer of hydrocolloid adhesive has a skin-facing surface and an opposite infusion hub-facing surface
wherein the release liner is provided on the skin-facing surface.

25. A method of adapting an infusion hub, to be suitable for use for an extended period of time of more than 7 days according to any one of claims 1 to 4, or 16 to 24; a kit of parts according to claim 5, or any one of claims 16 to 24; an infusion hub or a patch pump according to any one of claims 6 to 8, or 16 to 24; an infusion set according to any one of claims 9 or 16 to 24; an adaptor according to any one of claims 10 to 13, or 16 to 24; or a method of forming an adhesive structure according to any one of claims 14 to 24 30 wherein the opening extends through the entire adhesive structure, including the adhesive layer and the release liner.

26. A method of adapting an infusion hub, to be suitable for use for an extended period of time of more than 7 days according to claim 25; a kit of parts according to claim 25; an infusion hub or a patch pump according to claim 25; an infusion set according to claim 25; an adaptor according to claim 25; or a method of forming an adhesive structure according to claim 25 wherein the opening through the adhesive layer and release liner is a through-hole.

27. A method of adapting an infusion hub, to be suitable for use for an extended period of time of more than 7 days according to claim 25; a kit of parts according to claim 25; an infusion hub or a patch pump according to claim 25; an infusion set according to claim 25; an adaptor according to claim 25; or a method of forming an adhesive structure according to claim 25 wherein the opening through the adhesive layer is a through hole and the opening in the release liner is a slit.
